(19) United States
(12) Patent Application Publication
(10) Pub. No.: US 2010/0016981 A1
(43) Pub. Date: Jan. 21, 2010

(54) BONE REINFORCEMENT DEVICE

(75) Inventor: Gregory J. Roger, New South Wales (AU)

Correspondence Address: SCHWEGMAN, LUNDBERG & WOESSNER, P.A., P.O. BOX 2938, MINNEAPOLIS, MN, 55402, US

(73) Assignee: Advanced Surgical Design & Manufacture Limited, St., Leonards ,New South Wales (AU)

(21) Appl. No.: 12/374,893

(22) PCT Filed: Jul. 23, 2007
(86) PCT No.: PCT/AU2007/001022

§ 371 Date: Jul. 24, 2009

(30) Foreign Application Priority Data
Jul. 24, 2006 (AU) 2006903986

(51) Int. Cl. A61F 2/38; A61B 17/58

(52) U.S. Cl. 623/20.32; 606/86 R

(57) ABSTRACT

A reinforcement device (10) suitable for use with a prosthesis, such as a unicompartmental tibial component prosthesis. The reinforcement device is positionable between said component prosthesis and a surgically resected area of bone, for example, the tibia of a patient. The reinforcement device (10) includes a plate-like main body (12) extending from an anterior end (13) to a posterior end (14) and having an upper surface (16) and a lower surface (17). The lower surface (17) is engageable with a fixing means, such as a bone cement (25), to fix the tibial component to a surgically resected area of tibia. The main body (12) also has a plurality of perforations (15) therethrough.

### CROSS-REFERENCE TO RELATED APPLICATION

The present application claims priority from Australian Provisional Patent Application No 2006903986 filed on 24 Jul. 2006, the content of which is incorporated herein by reference.

### FIELD OF THE INVENTION

The present invention relates to a device for reinforcing an orthopaedic prosthesis. Particularly, the present invention relates to a device for improving the stiffness of cement underlying a polyethylene or ceramic component of a prosthesis including a tibial prosthesis and particularly the tibial component of a unicompartmental knee prosthesis.

### BACKGROUND ART

Orthopaedic prosthetic implants are subjected to substantial wear and tear and typically have a certain life, dependent upon a number of factors.

One major factor that affects the survival of a prosthetic implant is the condition of bone that supports the implant. The bone onto which an implant is mounted is typically of varying quality both from patient to patient and depending on the degree of resection. A deeper resection exposes spongy cancellous bone which does not provide a good support for overlying implants.

In many cases, the implant is cemented onto the bone such that the cement and the bone together form a layer that supports the implant during its life. The less stiff the layer (as is the case with a cancellous bone and cement support), the more flexing and bending of the implant, in particular an all polyethylene implant. Such flexing and bending leads to an increase in stresses in the polyethylene and reduces its life.

The device of the present invention aims to address the problems of the prior art.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention is a reinforcement device for use with an orthopaedic prosthetic component, said reinforcement device comprising a plate-like main body extending from an anterior end to a posterior end and having an upper surface and a lower surface, at least said lower surface engageable with a fixing means to fix the component to a surgically resected area of bone, wherein said main body further comprises a plurality of perforations therethrough and wherein said reinforcement device is positionable between said component and said surgically resected area of bone.

In a second aspect, the present invention is a reinforcement device for use with a unicompartmental tibial component prosthesis, said reinforcement device including a plate-like main body extending from an anterior end to a posterior end and having an upper surface and a lower surface, at least said lower surface engageable with a fixing means to fix the component to a surgically resected area of tibia, wherein said main body further comprises a plurality of perforations therethrough and wherein said reinforcement device is positionable between said component and said surgically resected area of tibia.

In a third aspect, the present invention is a reinforcement device for an orthopaedic prosthetic component, said reinforcement device comprising a plate-like main body extending from an anterior end to a posterior end and having an upper surface and a lower surface, at least said lower surface engageable with a fixing means to fix the component to a surgically resected area of bone, said main body further being comprised of at least an anterior region and at least a posterior region wherein the perforations of the posterior region differ in size and/or spacing and/or number relative to the perforations of the anterior region.

The survival or wear of the component of the orthopaedic prosthetic component of the first aspect and third aspect and the tibial component of the second aspect (hereinafter referred to as “component”) may depend upon the quality of material underlying the component when it is in situ in a patient.

Typically, the component is secured to the bone of a patient by fixing means. The fixing means is preferably cement which, in addition to fixing the component, may also provide a support layer together with the underlying bone to support the component. The cement may comprise a settable cement including an acrylic bone cement. Typically, the bone cement includes one or more additives such as an acrylic polymeric powder, such as polymethylmethacrylate (PMMA). The acrylic polymeric powder may be premixed with a liquid acrylic monomer system, which may include methyl methacrylate (MMA), resulting in a substance with a dough-like consistency, which is subsequently applied to the resected bone surface or to the reinforcement device. The bone cement may then be cured or polymerized and hardened in order to secure the prosthetic component within the bone.

The reinforcement device typically increases the stiffness of the cement. In this regard, the cement and any overlying component are subjected to various stresses in situ, including axial and torsional loads. If the cement has less strength than normal cortical bone (including tensile strength, shear strength and fatigue strength), it does not provide optimal support for an overlying component. In an embodiment wherein the component is a polymeric component, such as a polyethylene component, such lack of support may result in bending and warping of the component under normal physiological loads. The reinforcement member of the present invention thus increases the stiffness of the cement. Where the cement overlies a resected area of bone, the reinforcement member increases the stiffness of the cement/bone complex. The term “stiffness” is to be understood as including axial stiffness, torsional stiffness, shear stiffness and/or bending stiffness. Preferably, the stiffness of the cement or the cement/bone complex is increased by the presence of the reinforcement member towards a value of the stiffness of normal cortical bone.

The reinforcement device may, therefore, have particular application in cases where the area of bone upon which the component is to be implanted lacks sufficient stiffness to support the component long term. An example of such bone includes cancellous bone which is far more spongy and less stiff than cortical or subchondral bone. Even in cases where the component is supported by, for example, subchondral bone, the reinforcement device may still increase the stiffness of the cement and the bone support layer to thus improve the survival of the component when in use.

In one embodiment, the component and in particular the unicompartmental tibial component of the second aspect comprises an all polymeric component, for example an all polyethylene component. The increase in stiffness in the support layer may reduce flexing and bending of the polyethylene thus reducing the stresses in the polyethylene. Such a reduction in bending and flexing increases the life of the component.

The main body of the reinforcement device typically has a relatively flat upper surface and an opposite relatively flat lower surface. In this embodiment, the lower surface is configured such that it is receivable within a relatively flat resected area of bone, for example a resected area of a tibia including the lateral and/or the medial compartments of the tibia.

The main body may be between about 0.2 mm and 1 mm thick. Preferably, the main body is between about 0.3 mm and 0.7 mm thick and more preferably about 0.5 mm thick.

The main body is preferably made from a metal or a metal alloy. Preferably, the main body is made from surgical grade Chrome Cobalt alloy, titanium, a titanium alloy or stainless steel.

The main body of the reinforcement device may comprise a mesh member having a series of interconnecting strut members that define the perforations of the main body.

The perforations of the main body may be uniformly sized. Alternatively, and as defined in the third aspect, the size of at least some of the perforations may differ relative to at least some other perforations. In this embodiment, a portion of the main body having larger perforations may be less stiff than a portion of the main body which has smaller perforations. Further, the perforations may be uniformly spaced relative to each other in the main body. Alternatively, the perforation may be non-uniformly spaced. In this embodiment, the perforations of at least a portion of the main body may be relatively more spaced from each other than the perforation of another portion of the main body.

In one embodiment of the first and second aspects, the main body may comprise an anterior region and a posterior region. In these embodiments and in the third aspect, the posterior region may be relatively stiffer than the anterior region. Alternatively, the anterior region may be relatively stiffer than the posterior region.

In one embodiment, the posterior region of the main body may have relatively smaller perforations than the perforations of the anterior region. In another embodiment, the posterior region may have less perforations than the number of perforations of the anterior region. In a still further embodiment, the posterior region may have relatively more spaced apart perforations than the perforations of the anterior region.

The perforations may each comprise a passage extending from an opening in the upper surface to an opening in the lower surface. The openings in each surface may comprise a number of shapes including round, ovoid, square, triangular, diamond, rectangular and many other variations including combinations of these shapes.

The perforations of the main body may receive the bone cement. The lower surface of the reinforcement device may be brought into engagement with the bone cement such that the cement is forced through the perforations and up towards or beyond the upper surface of the reinforcement device. The cement may entirely cover the upper surface in this manner. Alternatively, the upper surface of the reinforcement device may receive the cement which may then flow through the perforations in the main body towards and beyond the lower surface of the device.

The lower surface of the main body may also be partially, substantially or wholly coated with an agent to enhance device/bone engagement. An example of a suitable agent is hydroxyapatite. The hydroxyapatite coating of this embodiment may optimise fixation of the main body to the underlying bone.

The reinforcement device may further include at least one spike member which depends from the lower surface of the main body. In use, the at least one spike member may be driven into the underlying bone to further secure the main body to said underlying bone. Typically, the reinforcement device includes a plurality of spikes. The spikes can be uniformly or non-uniformly distributed over the lower surface of the main body. In another embodiment, only a portion of the lower surface may have a plurality of spikes depending therefrom.

The reinforcement device may further include a detachable skirt member. The skirt member may extend upwardly and away from the outer edges of the main body that define the upper surface of the main body. Typically, the skirt member is made from a polymeric material, such as a polyethylene material. The skirt member may contain the bone cement during fixing of the reinforcement device to the underlying bone and fixing of the component to the reinforcement device. In use, once the component is in place, the skirt member may be detached from the main body of the reinforcement device and removed.

In use, the reinforcement member of the present invention may be driven or impacted onto an area of resected bone. Several holes may be punched into said bone during or after application of the reinforcement device. In one embodiment, the reinforcement device is impacted onto the bone and, once in situ, holes punched through the device and into the bone. Alternatively, the reinforcement device may be driven onto the bone by a holder member that also serves to simultaneously key holes into the underlying bone.

In the above embodiment, the upper surface of the reinforcement device may receive the cement which may then flow through the perforations of the reinforcement device and into the holes created in the underlying bone to provide a reinforced cement and bone layer to support the component.

In addition to, in one embodiment, being sized and shaped to generally correspond with a resected area of tibia, the reinforcement device may also generally correspond with the size and shape of the implant. The size and shape of the reinforcement device may vary, therefore, depending upon the size and shape of implant used.

In a fourth aspect, the present invention is a method of providing a support for a component of an orthopaedic prosthetic implant; said method including:
(i) surgically exposing an area of affected bone;
(ii) resecting an area of bone that will receive the component;
(iii) applying a reinforcement device to said area of bone, said reinforcement device comprising a plate-like main body that is sized and shaped to generally correspond with the surgically resected area of bone, said main body having an upper surface and an opposite lower surface and a plurality of perforations therethrough;
(iv) applying fixing means in a flowable state to the upper surface of the main body;
(v) causing or allowing the flowable fixing means to flow through the perforations of the main body from the upper surface to the lower surface to form a reinforcement device/fixing means combination;
(vi) applying the component of the orthopaedic prosthetic implant to the reinforcement device/fixing means combination formed by steps (iii) to (v).

In a fifth aspect, the present invention is a method of providing a support for a component of an orthopaedic prosthetic implant; said method including:
(i) surgically exposing an area of affected bone;
(ii) resecting an area of bone that will receive the component;
(iii) applying fixing means in a flowable state to the resected area of bone;
(iv) applying a reinforcement device to said fixing means, said reinforcement device comprising a plate-like main body that is sized and shaped to generally correspond with the surgically resected area of bone, said main body having an upper surface and an opposite lower surface and a plurality of perforations therethrough;
(v) causing or allowing the flowable fixing means to flow through the perforations of the main body from the lower surface to the upper surface to form a reinforcement device/fixing means combination;
(vi) applying the component of the orthopaedic prosthetic implant to the reinforcement device/fixing means combination formed by steps (iii) to (v).

The method of the fourth and fifth aspects may be used to provide a support for a tibial component of a unicompartmental prosthesis in a knee joint of a patient.

Several holes may be punched into the resected area of bone during or after positioning of the reinforcement device in vivo. In one embodiment, the reinforcement device is impacted onto the resected bone and, once in situ, holes punched through the device and into the bone. Alternatively, the reinforcement device may be driven onto the bone by a holder member that also serves to simultaneously key holes into the underlying bone.

In yet another aspect, the present invention consists in a reinforcement device for a component of an orthopaedic prosthesis, said reinforcement device including a main body that is sized and shaped to generally correspond with a surgically resected area of bone, said main body extending from an anterior end to a posterior end and having a plurality of perforations therethrough and wherein said reinforcement device is positionable between said orthopaedic prosthesis and said surgically resected area of bone and is fixable thereto by fixing means, the reinforcement device relatively increasing the stiffness of the fixing means.

In a still further aspect, the present invention consists in a reinforcement device for a unicompartmental tibial component prosthesis, said reinforcement device including a main body that is sized and shaped to generally correspond with a surgically resected area of tibia, said main body extending from an anterior end to a posterior end and having a plurality of perforations therethrough.

In these two further aspects, the mentioned features can have the features of the embodiments defined herein with respect to the first three aspects.

### BRIEF DESCRIPTION OF THE DRAWINGS

By way of example only, embodiments of the present invention are depicted in the attached drawings:

FIG. 1 is a schematic view of a knee joint of a patient fitted with the device of the present invention;

FIG. 2 is a schematic view of a resected tibia of a patient with the device of the present invention fitted;

FIG. 3 is a cross-sectional view of the device of the present invention when fitted to underlying bone;

FIG. 4 is a side elevational view of another embodiment of the invention; and

FIG. 5 is a side elevational view of a further embodiment of the invention.

### DETAILED DESCRIPTION OF AN EXEMPLARY EMBODIMENT OF THE PRESENT INVENTION

The following description relates to a reinforcement device that is used to reinforce the underlying cement and bone layer of a tibial component of a unicompartmental prosthetic implant. It will be appreciated that the reinforcement device may be used to reinforce the bone and cement underlying other types of orthopaedic prosthetic components.

The reinforcement device is generally depicted as 10 in the accompanying drawings. The reinforcement device 10 is used to increase the stiffness of the cement or the cement/bone underlying a unicompartmental tibial implant 50. The device is particularly useful in instances where the stiffness underlying the implant affects the wear and the ultimate survival of the implant such as the case in an all polyethylene implant. In this case, a compromised cement and bone layer beneath the implant leads to bending and flexing of the polyethylene which results in stresses therein.

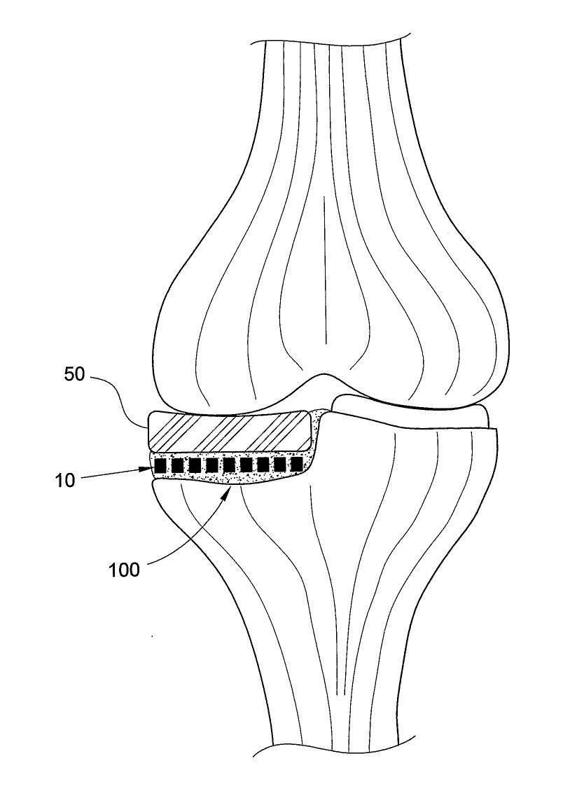

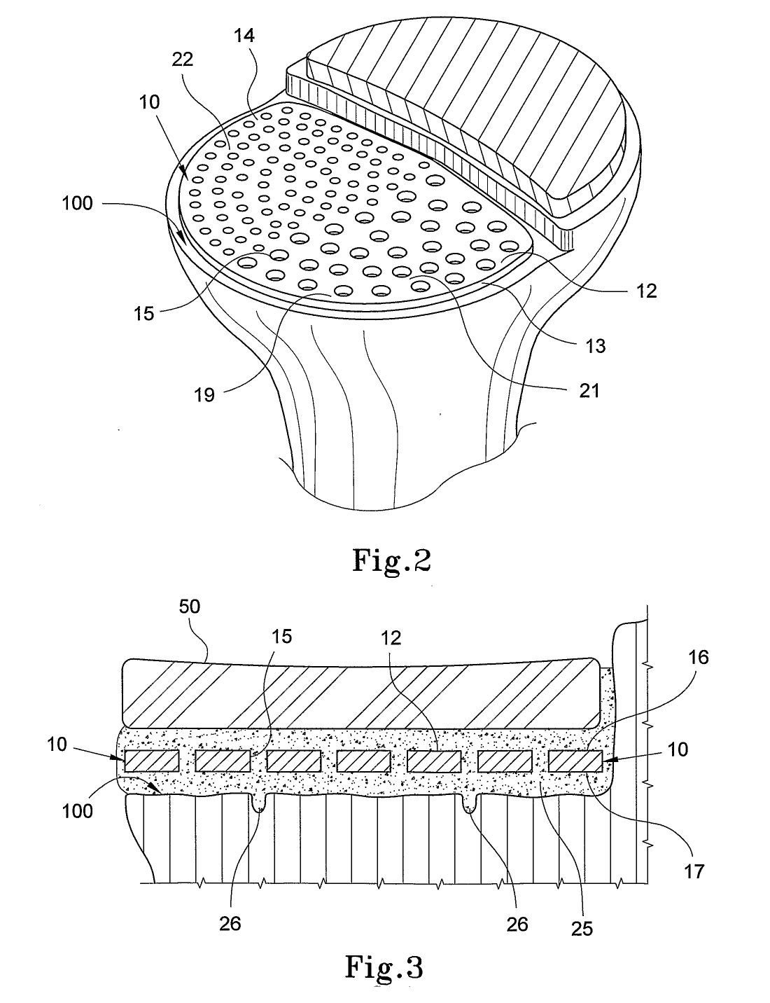

The reinforcement device 10 comprises a main body 12 that is sized and shaped to generally correspond with a surgically resected area of tibia 100 as shown in FIGS. 1 and 2. The main body 12 extends from an anterior end 13 to a posterior end 14. The main body 12 of the depicted embodiment has a plurality of perforations 15 therethrough.

The main body 12 has a relatively flat upper surface 16 spaced from a relatively flat lower surface 17.

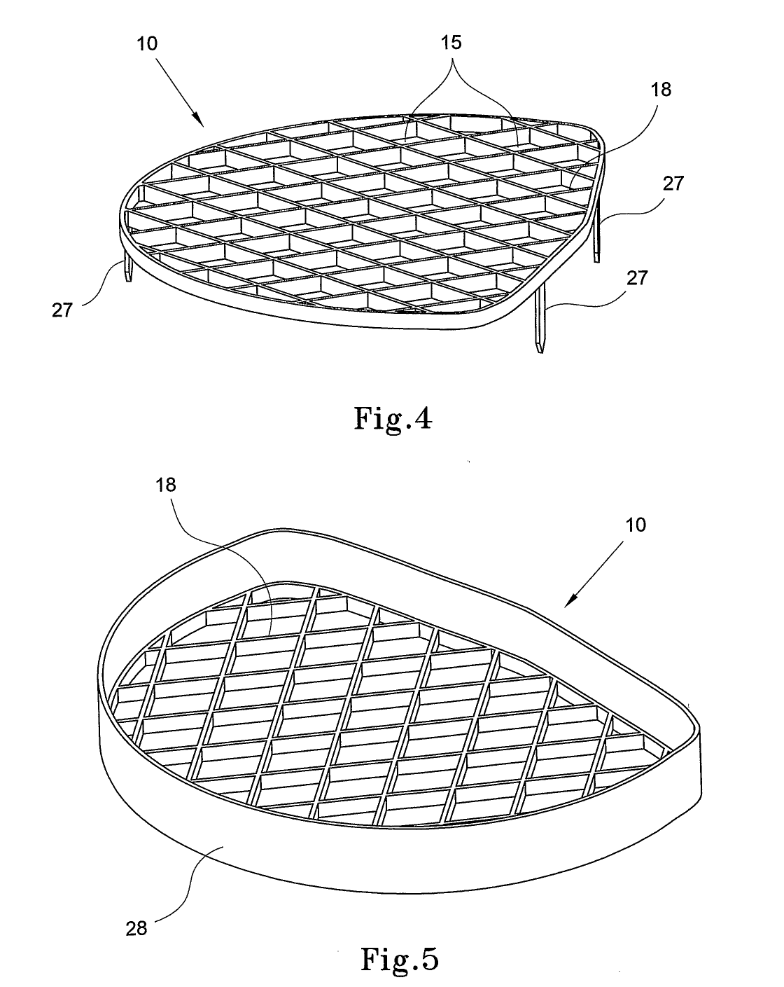

In the embodiment depicted in FIGS. 4 and 5, the main body 12 is a mesh member 18. In the embodiment depicted in FIG. 2, the main body 12 is a plate 19 that has pores therethrough. The perforations 15 of the mesh member 18 or plate 19 can vary in size and distribution across the main body. FIG. 2 depicts this variation with respect to the plate 19.

Plate 19 has an anterior region 21 and a posterior region 22. The perforations 15 of the posterior region 22 are smaller in diameter than the perforations 15 of the anterior region 21 providing a relatively stiffer posterior region of the main body 12.

The perforations 15 of the main body 12 receive bone cement 25 that fixes the main body 12 to the resected tibial surface 100. The bone cement 25 also secures a tibial implant 50 to the reinforcement device 10.

The reinforcement device 10 depicted in FIG. 4 has spike members 27 that depend from the lower surface 17 of the main body 12. The spike members 27 are driven into the underlying bone during impaction of the device onto the bone. The device 10 could have more spikes than that depicted with such spikes being uniformly or non-uniformly spaced. It will be appreciated that the plate 19 could also be provided with such spikes.

The reinforcement device 10 depicted in FIG. 5 has a detachable skirt member 28. The skirt member 28 extends upwardly and away from the upper surface 16 of the main body 12. The depicted skirt member 28 is made from a polyethylene material and contains the bone cement 25 during the initial stages of fixing the reinforcement device 10 to the underlying bone 100.

In use, a surgeon resects an area of tibia of a diseased compartment of the knee joint. The reinforcement device is then impacted onto the bone. The surgeon may then stamp holes 26 through the device an into the bone. Bone cement 25 is the applied which flows through perforations 15 and into holes 26 to provide a reinforced bone/cement layer combination.

The tibial implant 50 is then placed over the main body 12 and the joint of the patient extended to pressurise the implant, reinforcement device and the cement together and to the underlying bone.

The reinforcement device 10 increases the stiffness of the bone cement 25 and the underlying bone to optimally support the tibial implant 50.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A reinforcement device, said reinforcement device comprising a plate-like main body extending from an anterior end to a posterior end and having an upper surface and a lower surface, at least said lower surface engageable with a fixing means to fix the component to a surgically resected area of bone or tibia, wherein said main body further comprises a plurality of perforations therethrough and wherein said reinforcement device is positionable between said component and said surgically resected area of bone or tibia.

2. (canceled)

3. A reinforcement device for an orthopaedic prosthetic component, said reinforcement device comprising a plate-like main body extending from an anterior end to a posterior end and having an upper surface and a lower surface, at least said lower surface engageable with a fixing means to fix the component to a surgically resected area of bone, said main body further being comprised of at least an anterior region and at least a posterior region wherein the perforations of the posterior region differ in size and/or spacing and/or number relative to the perforations of the anterior region.

4. The reinforcement device of claim 1 wherein the fixing means is a settable cement.

5. The reinforcement device of claim 1 wherein the unicompartmental tibial component is an all polymeric component.

6. The reinforcement device of claim 1 wherein the main body has a relatively flat upper surface and an opposite relatively flat lower surface, the lower surface being configured such that it is receivable within a relatively flat resected area of bone.

7. The reinforcement device of claim 1 wherein the main body is between about 0.2 mm and 1 mm thick.

8. The reinforcement device of claim 1 wherein the main body is made from a metal or a metal alloy.

9. The reinforcement device of any one of claim 1 wherein the main body is a mesh member having a series of interconnecting strut members that define the perforations of the main body.

10. The reinforcement device of claim 1 wherein the perforations of the main body are uniformly sized.

11. The reinforcement device of any one of claim 1 wherein the size of at least some of the perforations differ relative to at least some other perforations.

12. The reinforcement device of claim 1 wherein the main body comprises an anterior region and a posterior region, wherein the posterior region is relatively stiffer than the anterior region or the anterior region is relatively stiffer than the posterior region.

13. The reinforcement device of claim 3 wherein: the posterior region has relatively smaller perforations than the perforations of the anterior region; and/or the posterior region has less perforations than the number of perforations of the anterior region; and/or the posterior region has relatively more spaced apart perforations than the perforations of the anterior region.

14. The reinforcement device of claim 1 wherein the perforations each comprise a passage extending from an opening in the upper surface to an opening in the lower surface, with the openings in each surface comprising a number of shapes including round, ovoid, square, triangular, diamond, rectangular and combinations of these shapes.

15. The reinforcement device of claim 1 wherein the lower surface of the main body is partially, substantially or wholly coated with an agent to enhance device/bone engagement.

16. The reinforcement device of claim 15 wherein the agent is hydroxyapatite.

17. The reinforcement device of claim 1 wherein the main body further includes at least one spike member which depends from the lower surface of the main body.

18. The reinforcement device of claim 1 wherein a detachable skirt member extend upwardly and away from the outer edges of the main body defining the upper surface of the main body.

19. The reinforcement device of claim 18 wherein the skirt member is made from a polymeric material.

20. A method of providing a support for a component of an orthopaedic prosthetic implant; said method including:
(i) surgically exposing an area of affected bone;
(ii) resecting an area of bone that will receive the component;
(iii) applying a reinforcement device to said area of bone, said reinforcement device comprising a plate-like main body that is sized and shaped to generally correspond with the surgically resected area of bone, said main body having an upper surface and an opposite lower surface and a plurality of perforations therethrough;
(iv) applying fixing means in a flowable state to the upper surface of the main body or the resected area of bone;
(v) causing or allowing the flowable fixing means to flow through the perforations of the main body from the upper surface to the lower surface to form a reinforcement device/fixing means combination;
(vi) applying the component of the orthopaedic prosthetic implant to the reinforcement device/fixing means combination formed by (iii) to (v).

21. (canceled)

22. The method of claim 20 further comprising providing a support for a tibial component of a unicompartmental prosthesis in a knee joint of a patient.
